(19) United States
(12) Patent Application Publication
(10) Pub. No.: US 2026/0033854 A1
(43) Pub. Date: Feb. 5, 2026

(54) POSITIONING AN INFLATABLE BLADDER INSIDE A BODY

(71) Applicant: BioProtect Ltd., Tzur Yigal (IL)

(72) Inventor: Shaul Shohat, Kfar Ha-Oranim (IL)

(21) Appl. No.: 19/283,167

(22) Filed: Jul. 28, 2025

Related U.S. Application Data
(60) Provisional application No. 63/678,990, filed on Aug. 2, 2024.

(51) Int. Cl. A61B 17/32; A61B 8/12; A61B 17/00; A61B 34/20; A61B 90/00

(52) U.S. Cl. CPC A61B 17/32; A61B 34/20; A61B 8/12; A61B 2017/00557; A61B 2017/320048; A61B 2034/2063; A61B 2090/3925

(57) ABSTRACT

A method of separating tissue masses includes inserting a portion of a tissue separation assembly between tissue masses inside a living body, by placing an inflatable bladder of the tissue separation assembly into an interface between the tissue masses with a tube fluidly coupled to the inflatable bladder. The tissue separation assembly includes a support extending away from the tube along one side of the inflatable bladder. The method also includes imaging a portion of the body to generate a visual representation that shows the support as visually distinguishable from the inflatable bladder. The method also includes adjusting an orientation of the inserted tissue separation assembly, based on the visual representation, to position the inflatable bladder. The method also includes inflating the inflatable bladder to separate the tissue masses, and then withdrawing the tube and support from the living body, leaving the inflatable bladder in place.

### CROSS-REFERENCE TO RELATED APPLICATIONS

This application claims the benefit of priority to U.S. Provisional patent Application No. 63/678,990, filed on Aug. 2, 2024, the entire contents of which are hereby incorporated by reference in its entirety.

### TECHNICAL FIELD

This disclosure relates to tissue separation devices, and more particularly to tissue separation devices used in ultrasound-guided tissue separation.

### BACKGROUND

Inter-tissue and inter-organ spacers are often used for creating and occupying a dissected tissue space in a human subject. Examples of such inflatable bladders or spacers can be found in U.S. patent application Ser. No. 11/918,414B2, which is incorporated herewith by reference in its entirety. Typically, such spacers are used to distance a healthy tissue or organ from another tissue or organ that is targeted for a treatment such as, for example, a radiation treatment. Spacers can also be used to separate tissue in other surgical procedures, such as in laparoscopic surgery and endoscopic procedures. In radiation therapy, the tissue dissection process and spacer placement can reduce the exposure of the healthy tissue to the potentially negative effects of the treatment. The spacers are often introduced into the tissue spaces while the surgeon is guided by ultrasound imaging. Accurately positioning the spacer in an optimal position and location can be difficult. Methods and equipment to improve tissue separation and spacer placement procedures are sought.

### SUMMARY

Implementations of the present disclosure include a method of separating tissue masses. The method includes inserting a portion of a tissue separation assembly between tissue masses inside a living body, by placing an inflatable bladder of the tissue separation assembly into an interface between the tissue masses with a tube fluidly coupled to the inflatable bladder. The tissue separation assembly includes a support coupled to the tube and extending away from the tube along one side of the inflatable bladder. The method also includes imaging a portion of the body to generate a visual representation of an internal portion of the body. The visual representation shows the support as visually distinguishable from the inflatable bladder. The method also includes adjusting an orientation of the inserted tissue separation assembly, based on the visual representation, to position the inflatable bladder. The method also includes inflating the inflatable bladder to at least partially separate the tissue masses. The method also includes withdrawing the tube and support from the living body, leaving the inflatable bladder in place.

In some implementations, the support includes a visual marker and the adjusting includes adjusting, using the visual marker as a reference point in the visual representation, the position of the inflatable bladder to center the inflatable bladder with respect to the tissue masses.

In some implementations, the support is movable radially along a first axial plane of the tube and fixed laterally along a second axial plane of the tube orthogonal with respect to the first axial plane. The support is releasably coupled to the inflatable bladder and configured to be displaced along the first axial plane with the inflatable bladder as the inflatable bladder is inflated. Adjusting the orientation of the tissue separation assembly includes moving the tube to move the support and the inflatable bladder with the tube as the support and the inflatable bladder are aligned with the first axial plane.

In some implementations, the adjusting includes moving the inflatable bladder together with the support while the support restricts a lateral displacement of the inflatable bladder along the second axial plane. The support allows the inflatable bladder to inflate and expand along the first axial plane and the second axial plane.

In some implementations, the support includes a proximal end attached to the tube and a distal end releasably coupled to an end of the inflatable bladder. The adjusting includes moving the inflatable bladder while the support maintains a radial orientation of the inflatable bladder with respect to a central axis of the tube.

In some implementations, the support is made of a metal visible in ultrasound imaging and X-ray imaging, and the imaging includes ultrasound imaging or X-ray imaging.

In some implementations, the imaging includes generating an axial view of the tissue masses utilizing a transrectal ultrasound probe. The axial view shows a cross-section orthogonal with respect to a central longitudinal axis of the transrectal ultrasound probe.

In some implementations, the support has a non-symmetrical cross-section, and the adjusting includes adjusting, using the non-symmetrical cross-section as a reference, the position of the support to adjust the position of the inflatable bladder.

In some implementations, adjusting the orientation of the inserted tissue separation assembly includes adjusting, after the inflatable bladder is fully inflated, the inserted tissue separation to position the inflatable bladder.

In some implementations, the tissue masses include soft tissue masses that reside in an anatomic target location inside the body, and the method further includes, after inflating the inflatable bladder, withdrawing the tube and the support from the body, leaving the inflatable bladder in the anatomic target location inflated inside the body. In some implementations, the anatomic target location includes a location between prostate tissues and rectum tissues.

In some implementations, the imaging a portion of the body includes generating the visual representation on an electronic screen.

In some implementations, the support includes an elongated arm having one end attached to the tube, with the arm extending parallel to the tube across at least a portion of the inflatable bladder, and the adjusting includes adjusting the orientation of the inserted tissue separation assembly by moving the tube as the arm remains parallel to the tube and attached to the tube and the inflatable bladder.

In some implementations, before adjusting the orientation of the inserted tissue separation assembly, the method includes inflating the inflatable bladder to a partially inflated state. The adjusting includes adjusting, with the inflatable bladder in the partially inflated state, the position of the inflatable bladder, and inflating the inflatable bladder to at least partially separate the tissue masses includes inflating the inflatable bladder to a fully inflated state.

In some implementations, the method further includes, before withdrawing the tube and support from the living body, determining, based on the visual representation, that the inflated inflatable bladder is not in the correct position. The method also includes deflating the inflatable bladder. The method also includes readjusting, based on the visual representation, the position of the inflatable bladder. The method also includes reinflating, with the orientation of the inflatable bladder adjusted, the inflatable bladder.

In some implementations, the tissue separation assembly includes a second support, the imaging includes generating the visual representation showing the second support as visually distinguishable from the inflatable bladder and the support, and the adjusting includes adjusting the position of the inflatable bladder based on a location of the support and the second support in the visual representation.

In some implementations, the method further includes, before withdrawing the tube and support from the living body, sealing the inflatable bladder inside the living body.

Implementations of the present disclosure include a tissue separation assembly that includes a tube, and inflatable bladder, and a support. The inflatable bladder is releasably attached and fluidly coupled to the tube. The support is coupled to the tube and extends away from the tube along a side of the inflatable bladder. The inflatable bladder is inserted by the tube between tissue masses inside a living body into an interface between the tissue masses. The support is made of a material visually distinguishable from the inflatable bladder in a visual representation generated from body imaging of the living body, allowing an orientation of the inflatable bladder to be adjusted based on a position of the support in the visual representation.

In some implementations, the support is made of stainless steel. In some implementations, the support is made of opaque stainless steel.

In some implementations, the support includes an elongated arm that extends from the tube to a distal end of the inflatable bladder. The support is inserted into a ring of the inflatable bladder disposed at the distal end of the inflatable bladder so that the support maintains the distal end of the inflatable bladder aligned with the tube.

In some implementations, the support is movable radially in a direction parallel to a first axial plane of the tube and fixed laterally along a second axial plane of the tube orthogonal with respect to the first axial plane. The support is releasably coupled to the inflatable bladder and configured to be displaced, with the inflatable bladder, along the first axial plane as the inflatable bladder is inflated, allowing the inflatable bladder to be moved, in an inflated or deflated state, with the support to adjust a location of the inflatable bladder.

In some implementations, the support includes a proximal end attached to the tube and a distal end releasably coupled to an end of the inflatable bladder. The support allows the moving the of inflatable bladder while the support maintains a radial orientation of the inflatable bladder with respect to a central axis of the tube.

In some implementations, the support includes an elongated arm having one end attached to the tube. The arm extends parallel to the tube across at least a portion of the inflatable bladder.

In some implementations, the tissue separation assembly includes a second support at least partially spaced from the support and extending away from the tube along the side of the inflatable bladder. The second support is made of a material visually distinguishable from the inflatable bladder and from the support in a visual representation generated from body imaging of the living body, allowing an orientation of the inflatable bladder to be adjusted based on the position of the support and a position of the second support in the visual representation.

In some implementations, the inflatable bladder includes a self-sealing port through which the tube is inserted into the inflatable bladder. The self-sealing port automatically seals the inflatable bladder upon withdrawing the tube from the inflatable bladder.

Implementations of the present disclosure include a method of separating tissue masses. The method including inserting, with a tube fluidly coupled to an inflatable bladder, the inflatable bladder into an interface between tissue masses of a body. The method also includes imaging a portion of the body to generate a visual representation of an internal portion of the body. The visual representation shows a visual marker as visually distinguishable from the inflatable bladder. The visual marker is coupled to and extends from the tube over a side of the inflatable bladder. The method also includes adjusting, based on the visual representation, an orientation of the tube to position the inflatable bladder. The method also include inflating the inflatable bladder to at least partially separate the tissue masses. The method also includes withdrawing the tube and visual marker from the living body, leaving the inflatable bladder in place.

In some implementations, the visual marker includes a support that moves, with the inflatable bladder as the inflatable bladder is inflated, parallel to a first axial plane of the tube, and is fixed against movement along a second axial plane of the tube orthogonal with respect to the first axial plane.

Particular implementations of the subject matter described in this specification can be implemented so as to realize one or more of the following advantages. For example, the tissue separation assembly of the present disclosure has a support that serves as a marker, which can facilitate the positioning of the inflatable bladder at a desired or optimal location during ultrasound-guided procedures. The spacer also retains the inflatable bladder in one direction while allowing the inflatable bladder and retainer to be displaced in another direction, allowing the bladder to be, based on a location of the spacer in ultrasound imaging, inflated and placed in an optimal position.

### BRIEF DESCRIPTION OF THE DRAWINGS

FIG. 1 is a perspective, schematic view of a tissue separation assembly according to a first implementation of the present disclosure, with its inflatable bladder inflated.

FIG. 2 is a perspective, schematic view of the tissue separation assembly of FIG. 1, with its inflatable bladder deflated.

FIG. 3 is a top, schematic view of the tissue separation assembly of FIG. 1.

FIG. 4 is a top, schematic view of a tissue separation assembly according to a second implementation of the present disclosure.

FIGS. 5 and 6 show sequential steps of a method of using a tissue separation assembly.

FIG. 7 is a visual representation generated using body imaging, in which the inflatable bladder is in a correct position.

FIG. 8 is a visual representation generated using body imaging, in which the inflatable bladder is not in a correct position.

FIG. 9 is a back, cross-sectional, schematic view of a tissue separation assembly according to a third implementation of the present disclosure.

FIG. 10 is a flow chart of an example method of placing an inflatable bladder inside a living body.

### DETAILED DESCRIPTION OF THE DISCLOSURE

Apparatuses and methods for separation or dissection of one tissue from another are disclosed. The separation can be accomplished by delivering a spacer, such as an inflatable balloon or bladder, to reside between tissues until the bladder biodegrades and/or is removed. Such inflatable bladders are known to be useful in cases where physical separation between adjacent tissues, such as organs or organ tissues, is desirable. For example, an inflated bladder can help protect one tissue from effects of a treatment to the second tissue—for example, a radiation treatment. In some aspects, an apparatus for use in the separation procedures includes a tissue separation assembly with a marker adjacent the bladder to facilitate positioning the bladder at a desired or optimal location. A tissue separation assembly can be inserted into a subject's body, for example, through an incision in the subject's perineum or abdominal wall, depending on which tissues or organs are involved.

The tissue separation assembly can be inserted between tissue masses inside a living body by placing, with a tube attached to the bladder, the bladder into an interface between the tissue masses. The tissue separation assembly has a support in the form of an elongated arm that serves as a marker during imaging-guided tissue separation or dissection procedures. The support is coupled to the tube and extends away from the tube along one side of the inflatable bladder. Ultrasound or X-ray imaging of the body generates a visual representation of an internal portion of the body and shows the support as visually distinguishable from the inflatable bladder. The orientation of the inserted tissue separation assembly can be adjusted based on the visual representation to position the inflatable bladder at a desired position and location. Once the inflatable bladder is in the correct position, the bladder is inflated to separate the tissue masses. The tube and support can be withdrawn from the living body after sealing the bladder, leaving the inflatable bladder in place.

In some aspects, the tissue separation assembly includes a dilator or dilator assembly and a guide needle or inflation lumen inserted within the dilator. The dilator assembly can include a dilator and a dilator-sheath mounted concentrically around the dilator. The marker and inflatable bladder can be disposed inside the dilator. Once located at a target location, the bladder can be passed through the interior of the dilator sheath and deployed at or beyond the distal end of the dilator sheath. The marker can be used to position the dilator at the right location, as well as to position the inflatable bladder in the optimal location.

The locating of the bladder at the desired or optimal target location is accomplished by using the support as a marker (e.g., a hyperechoic marker or echogenic marker). The marker is arranged so as to be easily seen by a surgeon, e.g., on a monitor or other imaging device in communication with an ultrasound probe. The materials and shape of the marker can be selected to be more easily “seen” in sufficiently high contrast to the surrounding surface of the inflatable bladder to the marker, e.g., by ensuring that the acoustic impedance of the marker (e.g., the echogenicity of the marker) is significantly greater than the acoustic impedance of the surface area of the inflatable bladder adjacent the marker. For example, a metal or metal-alloy marker can be suitable for use with an inflatable bladder of a polymeric material such as, in a non-limiting example, biodegradable polyester.

With a higher acoustic impedance than the surrounding inflatable bladder, the marker can be helpful in accurately locating the bladder with respect to the marker, and then positioning the bladder at a desired or optimal location. In some aspects, the support substantially prevents lateral or side-to-side movement of the bladder, while allowing the bladder to expand radially as the bladder is inflated, allowing the support and the bladder to move radially together for better bladder positioning and orientation during ultrasound-guided procedures.

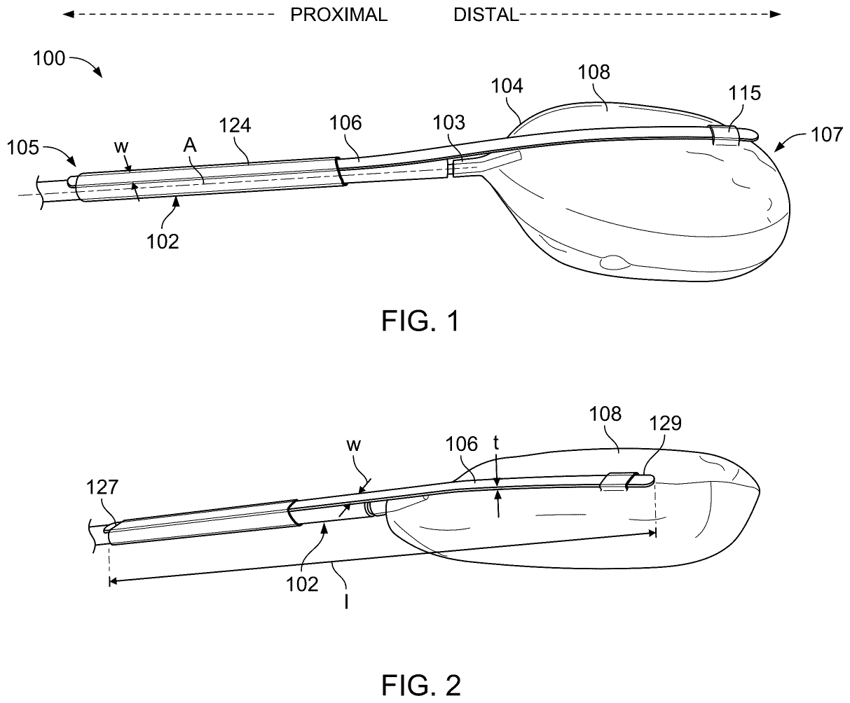

FIG. 1 shows a tissue separation assembly 100 that includes a tube 102 (e.g., a tube assembly), an inflatable bladder 104, and a support 106. The inflatable bladder 104 is releasably attached and fluidly coupled to the tube 102. The support 106 is attached to the tube 102 and extends away from the tube 102 along a side 108 of the inflatable bladder 104.

In some aspects, the support 106 is shaped as an elongated arm 106 that extends from the tube 102 to a distal end 107 of the inflatable bladder 104. For example, referring also to FIG. 2, the support 106 can have a “popsicle stick” shape, with rounded ends 127, 129, a length “1,” and a width “w” greater than its thickness “t.” The length “1” can be, for example, between 1 and 15 inches, the width “w” can be, for example, between 0.1 and 0.5 inches, and the thickness “t” can be, for example, between 0.01 and 0.1 inches.

The support 106 is attached to the tube 102 at a proximal end 105 of the support 106 and releasably attached to the inflatable bladder 104 at a distal end 107 of the support 106 and/or bladder 104. For example, the support 106 is inserted into a ring 115 of the inflatable bladder 104 that resides at the distal end 107 of the inflatable bladder 104 so that the support 106 prevents the distal end 107 (and by extension, the inflatable bladder 104) from moving from side to side (or from moving to the least “resistant to dissection” side), maintaining the bladder 104 generally aligned with the tube 102.

The terms ‘distal’ and ‘proximal’ as used throughout this disclosure and in the claims appended thereto are to be understood according to their accepted usage, wherein the ‘distal’ direction is the direction further into a patient's body and away from a user, e.g., a medical practitioner using the device, while the proximal direction indicates the opposite direction. Distal and proximal directions are shown for clarity in FIG. 1, with distal being to the right of the page, and proximal being to the left of the page.

The support 106 is movable with the inflatable bladder 104 in one direction, while fixed and not movable in a second direction. For example, the support 106 is flexible or bendable radially to move with the inflatable bladder 104 as the inflatable bladder 104 is inflated. Referring also to FIG. 2, with the inflatable bladder 104 deflated (or partially inflated), the support 106 is in a first position. In the first position, the support 106 is its original position or unbent position, meaning that the support 106 is not under bending stress applied by the inflatable bladder 104. As shown in FIG. 1, with the inflatable bladder 104 inflated, the support 106 is in a second position. In the second position, the support 106 is in a bent or flexed position, meaning that the support 106 is under bending stress applied by the inflatable bladder 104 pushing the support 106 away from the tube 102. In some aspects, the support 106 is radially movable with the bladder by pivoting about the tube 102, instead of bending or flexing.

The support 106 is made of a material that is visible in visual representations generated using body imaging. In other words, the support 106 is made of a material visually distinguishable from the inflatable bladder 104 in a visual representation generated from body imaging of a living body. For example, the support 106 is made of metal, plastic, or a composite, and has an opaque finish that allows the support 106 to be seen in ultrasound imaging or X-ray imaging. In some aspects, the support 106 is made of stainless steel, opaque stainless steel, or sandblasted stainless steel. Additionally, the support 106 or the finishing of the support can be formed, for example, by etching, engraving, cutting, chipping, rubbing, scraping, rasping, or abrading the elongated arm.

The inflatable bladder 104 can be made of a biodegradable material, such as biodegradable polymers. In some aspects, the inflatable bladder 104 is designed to remain inflated on site for weeks or months and fade away or dissolve within a number of weeks or months. Once at the desired location, the bladder 104 is inflated by, for example, injecting a fluid (e.g., a saline solution) with a syringe connected to the opposite end (e.g., the proximal side) of the tube 102 (or to a tube inside the tube).

The inflatable bladder 104 protects the tissues from radiation, mechanical stress, or other potential harms. In some aspects, the inflatable bladder 104 protects the normal tissues by increasing the gap between a radiation source and critical structures. The radiation can fade away while passing through the inflated bladder 104, creating a barrier that protects tissue from harmful radiation. Biodegradable inflatable balloons can also be used to isolate tissue for other purposes, such as to protect tissue from mechanical, thermal, or electrical stresses.

The inflatable bladder 104 has a port 103 through which the tube 102 is inserted to inflate the bladder 104. In some aspects, the port 103 is sealed or closed automatically when the tube 102 is withdrawn from the inflatable bladder 104. For example, the port 103 is a self-sealing port that allows the inflatable bladder 104 to remain inflated for a period of time after the tube 102 has been withdrawn. The self-sealing port 103 can be in the form of a plug located on the distal end of the inflation tube inside the bladder and seals the bladder once the tube is withdrawn/pulled out. The self-sealing port 103 can be in the form of an elastic port with adhesive that closes when the tube 102 is pulled out. In some aspects, the self-sealing port 103 has an elastic ring around the port 103 that closes the port when the tube 102 is pulled out. In some cases, the self-sealing port 103 has a valve such as a one-way valve (e.g., a flapper valve) that closes when the tube 102 is withdrawn. In some aspects, the self-sealing port is closed by viscosity of a liquid (e.g., one that forms a gel). The port 103 can also be manually sealed by, for example, clamping the port, applying external compression to the port 103, or knotting of the port 103. In some aspects, the port 103 is sealed before withdrawing the tube 102 and support 106 from the living body, sealing the inflatable bladder 104 inside the living body.

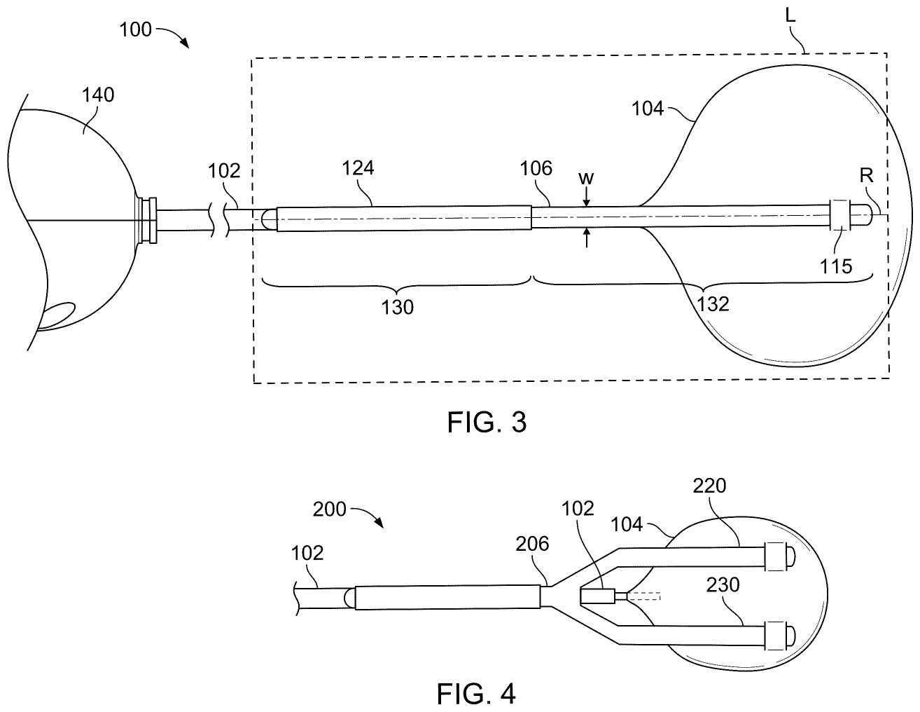

FIG. 3 is a top view of the tissue separation assembly 100. In some aspects, the separation assembly 100 includes a handle assembly 140 (shown in part) from which the tube 102 extends. The handle assembly can include a gripping surface that the user of the separation assembly 100 can grab to manipulate the separation assembly 100 during the procedure. The handle assembly 140 can also have apertures that allow tubes (e.g., syringes) to be inserted inside the tube 102.

The tube 102 defines a first axial plane “R” that extends across the longitudinal central axis “A” (shown in FIG. 1). The tube 102 also defines a second axial plane “L” across the longitudinal central axis “A.” The second axial plane “L” is orthogonal with respect to the first axial plane “R.” The first axial plane “R” extends through the middle of the support 106 and is parallel with respect to the thickness “t” (shown in FIG. 2) of the support 106. The second axial plane “L” extends through the middle of the tube 102 and is parallel with respect to a width “w” of the support 106.

The support 106 is bendable radially in a direction parallel to the first axial plane “R” and is fixed (or substantially fixed) laterally along the second axial plane “L.” Specifically, a proximal section 130 of the support 106 is secured to the tube 102 and a distal section 132 of the support 106 is not secured to the tube 102. The proximal section 130 of the support 106 is not movable with respect to the tube 102. However, the distal section 132 of the support 106 is movable (e.g., bendable) with respect to the tube 102 in the radial direction along the first axial plane “R.” The distal section 132 of the support 106 is not movable or bendable in the lateral direction (e.g., from side to side) with respect to the tube 102, along the second axial plane “L.” In other words, the support 106 is rigid in the direction of its width “w” and is secured to the tube 102, preventing the support 106 from bending or moving along the second axial plane “L” with respect to the tube 102.

The proximal section 130 of the support 106 can be attached to the tube 102 through a band 124 or brace (e.g., a heat shrink strip), or by other mechanical means such as fasteners, welded joints, etc. The distal section 132 of the support 106 expends over the inflatable bladder 104 to bend, with the bladder as the bladder expands, in the radial direction along the first axial plane “R.” The support 106 extends parallel to the tube 102 across a portion of the inflatable bladder 104 to maintain the inflatable bladder 104 aligned with the tube 102.

The support 106 is releasably coupled to the inflatable bladder 104 to prevent lateral displacement of the inflatable bladder 104 with respect to the support 106. For example, the distal end or section 132 of the support 106 is inserted into the ring 115 of the inflatable bladder 104 to retain the inflatable bladder 104 and prevent the inflatable bladder 104 from substantially moving along the axial plane “L” with respect to the support 106 and the tube 102. The support 106 allows the inflatable bladder 104 to expand or be inflated in any direction, including along the two axial planes “R” and “L.” The support 106 is able to be released from the inflatable bladder 104 by, for example, pulling the support 106 with the tube 102 away from the inflated bladder 104, causing the support 106 to be pulled out of the ring 115.

The support 106 is arranged to be displaced, with the inflatable bladder 104, along the first axial plane “R” as the inflatable bladder 104 is inflated. The support 106 maintains the radial orientation of the inflatable bladder 104 with respect to the central axis “A” (see FIG. 1) of the tube 102. This allows the inflatable bladder to inflate and move in the radial direction along with the support 106, while preventing the inflatable bladder 104 from moving in the lateral direction so that the location of the inflatable bladder 104 can be adjusted by moving the tube 102 from outside the living body.

FIG. 4 is a top view of a tissue separation device 200 according to a different implementation. The tissue separation device 200 has a two supports 220, 230. The first support 220 and a second support 230 have a function similar to the function of the support 106 in FIGS. 1-3, with the main exception that, in the visual representation of the tissue separation device 200, the imaging shows two supports 220, 230 to further aid the placement of the inflatable bladder 104. The second support 230 is spaced from the first support 220 and extends away from the tube 202 along the side of the inflatable bladder 104. The two supports 220, 230 are made of a material visually distinguishable from each other and from the inflatable bladder 104 in the visual representation generated from body imaging of the body, allowing an orientation of the inflatable bladder 104 to be adjusted based on the position of the two supports 220, 230 in the visual representation. In some aspects, the two supports 220, 230 are arms that extend from a support section 206 (e.g., an elongated bar) attached to the tube 102.

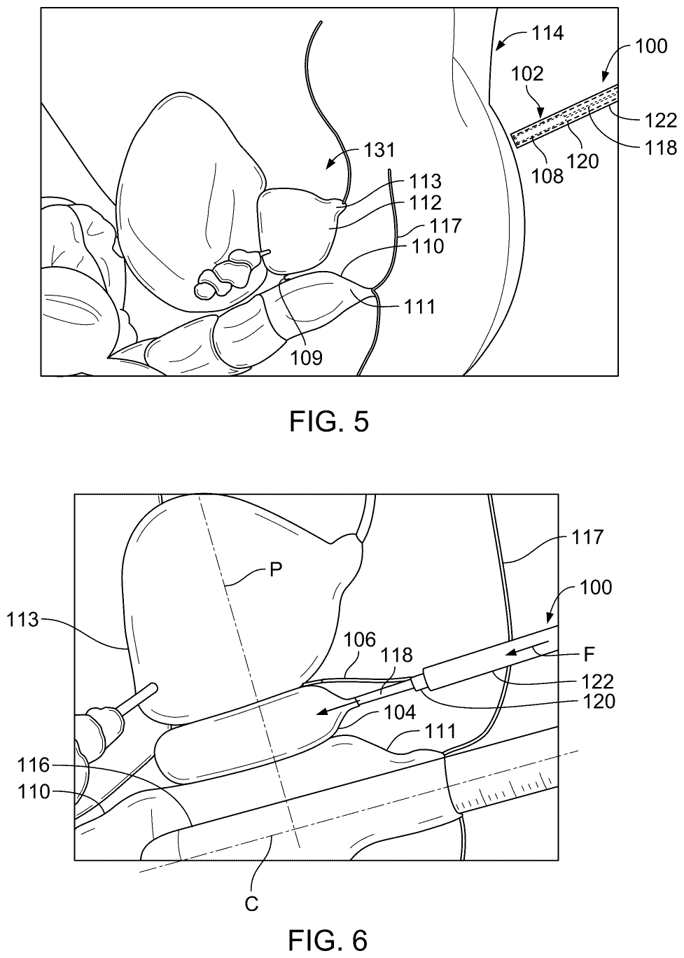

FIGS. 5 and 6 show sequential steps of a method of using the tissue separation assembly 100. To place the inflatable bladder inside a living body 114, the tissue separation assembly 100 is inserted into the body 114 through a perineum 117 of the body 114. The exemplary use case illustrates features that are applicable to other examples of tissues and organs as discussed herein and are not limited to the prostate-rectum case.

As shown in FIG. 5, the inflatable bladder 104 can reside within the tube 102 before the tube 102 is inserted into the body 114. Specifically, the tube 102 (e.g., an introducer tube or introducer tube assembly) can include multiple tubes, lumens, or needles. For example, the tube 102 includes a dilator sheath 122, a packaging sheath 120, and an inflation lumen 118 or needle. The packaging sheath 120 can be disposed within and be concentric with respect to the dilator sheath 122, and the inflation lumen 118 can be disposed within and be concentric with respect to the packaging sheath 120. The inflatable bladder 104 can be inserted into the body 114 in a deflated or partially deflated state using the dilator sheath 122, packaging sheath 120, and inflation lumen 118. For example, the inflatable bladder 104 can be rolled up, folded, bended, or otherwise deflated and inserted into the packaging sheath 120 before the inflatable bladder 104 is deployed into the body 114.

The dilator sheath 122 forms the outer housing of the tube 102 and houses the packaging sheath 120 and inflation lumen 118. The inflation lumen 118 is fluidly coupled to the inflatable bladder 104 to direct fluid into the inflatable bladder 104 to inflate the inflatable bladder 104. In some cases, the inflation lumen 118 receives a needle that directs the fluid into the inflatable bladder 104. In some aspects, the inflatable bladder 104 and the inflation lumen 118 are inserted into (or advanced along) the dilator sheath after the dilator sheath 122 has been inserted into the body 114.

The inflatable bladder 104 is arranged to be inserted by the tube 102 between tissue masses 110, 112 inside the body 114. More specifically, the inflatable bladder 104 can be positioned within an interface 109 defined between a first tissue mass 110 and a second tissue mass 112. In some aspects, the first tissue mass 110 is the soft tissue of a rectum 111 and the second tissue mass 112 is the soft tissue of a prostate 113. The tissue masses 110, 112 reside in an anatomic target location 131 inside the body 114. As shown in FIG. 6, the inflatable bladder 104 stays in the anatomic target location 131 in an inflated state, between the prostate 113 and the rectum 111.

The inflatable bladder 104 can be introduced using minimally invasive techniques (e.g., using a “key hole” introduction technique). For example, the inflatable bladder 104 is introduced using a minimally invasive method through the perineum 117 under transrectal ultrasound (TRUS) guidance. The inflatable bladder 104 can be collapsed to a miniature size, and then deployed by expanding or spreading the device in a highly controllable fashion in specific sizes and directions, thereby avoiding harm to adjacent organs and tissues, while performing the dissection or separation of tissue. The prostate 113 and surrounding tissues, as well as the space between rectum 111 and prostate 113, can be visualized using such modalities such as Trans-Rectal Ultrasound (TRUS), X-ray imaging, magnetic resonance imaging (MRI), or computed tomography (CT).

As seen in FIG. 6, a transrectal ultrasound probe 116 can be deployed for guiding the procedure. The probe 116 can be inserted into the rectum 111 of the body 114 before the tissue separation assembly 100 is inserted into the body 114. In FIG. 6, the tube 102 has been inserted through an incision in the subject's perineum 117 until the distal tip of the dilator sheath 122 reaches or is nearby the interface 109 between the rectal wall and the prostate 113. In some aspects, the positioning of the dilator sheath 122 can be guided based on ultrasound imaging showing the position of the support 106. Once the distal tip of the dilator sheath 122 is at the desired location (e.g., at the interface), the inflation lumen 118 is advanced (e.g., distally advanced) out of the dilator sheath 122 and packaging sheath 120. Alternatively, the dilator sheath 122 can be withdrawn (pulled back) while maintaining the inflatable bladder 104 and inflation lumen 118 in place so that the inflatable bladder 104 is maintained between the prostate 113 and the rectum wall.

After the inflatable bladder 104 is outside the dilator sheath 122 and the packaging sheath 120, fluid is flowed into the inflatable bladder 104 to inflate the bladder 104. The fluid “F” can be, for example, a saline solution or carbon dioxide gas. The fluid “F” can be flowed directly through the inflation lumen 118 or a syringe (e.g., a saline syringe) can be introduced through the interior of the inflator lumen 118 to inject fluid and inflate the inflatable bladder 104. Examples of such methods of deploying expandable separators can be found in PCT Publication No. WO2023144703, which is incorporated herewith by reference in its entirety.

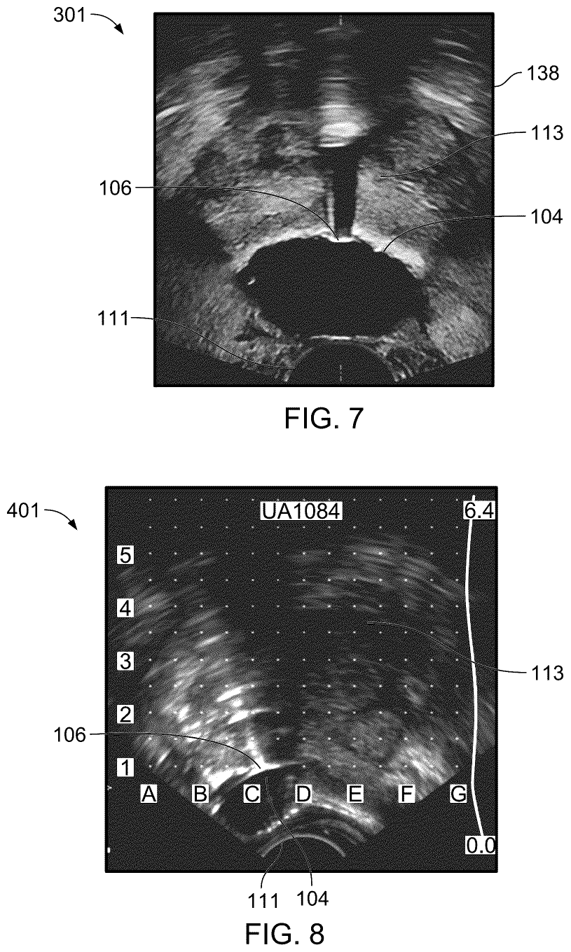

FIG. 7 shows a visual representation 301 of a portion of the human body 114, with the inflatable bladder 104 positioned correctly. In this example, the visual representation 301 is an axial ultrasound view. The visual representation 301 can be generated on an electronic screen 138. The support 106 is visually distinguishable from the inflatable bladder 104 in the visual representation 301. The visual representation 301 can be generated utilizing the transrectal ultrasound probe 116, as shown in FIG. 6. The visual representation 301 shows an image of a cross-section taken along plane “P,” which is orthogonal with respect to a central longitudinal axis “C” of the transrectal ultrasound probe 116. As shown in FIG. 7, the visual representation 301 shows the cross-section of the inflatable bladder 104 as a dark oval, and the cross-section of the support 106 as a bright, elongated spot adjacent and visually distinguishable from the inflatable bladder 104. Because the support 106 is visible in the visual representation 301, a user can adjust the orientation of the inflatable bladder 104 based on the position of the support 106 in the visual representation 301. Thus, the support 106 serves as a visual marker that can be used as a reference to move the inflatable bladder 104.

For example, while imaging the body, a medical practitioner using the tissue separation assembly 100 can move the inflatable bladder 104 based on the position of the support 106 with respect to the inflatable bladder 104. Because the support 106 remains at the center of the bladder 104, moving the support 106 to the center between the prostate 113 and the rectum 111 moves the inflatable bladder 104 to the center between the prostate 113 and the rectum 111. Thus, the user can adjust, based on the position of the support 106 in the visual representation 301, an orientation of the inserted tissue separation assembly to position the inflatable bladder 104 at the optimal position and orientation.

FIG. 8 shows a second visual representation 401 of a portion of the human body 114, with the inflatable bladder 104 positioned incorrectly. The visual representation 401 is also an axial ultrasound view, taken along plane “P.” The support 106 is bright as opposed to the dark inflatable bladder 104, and thus visually distinguishable from the inflatable bladder 104 in the visual representation 301. As shown in FIG. 8, the visual representation 301 shows the inflatable bladder 104 on a side of the rectum 111 and the prostate 113. Specifically, both the support 106 and the inflatable bladder 104 are shown to the right of the page. A practitioner manipulating the inflatable bladder can move the tube to the right of the page to center the support 106 and inflatable bladder 104 with respect to the rectum 111 and prostate 113.

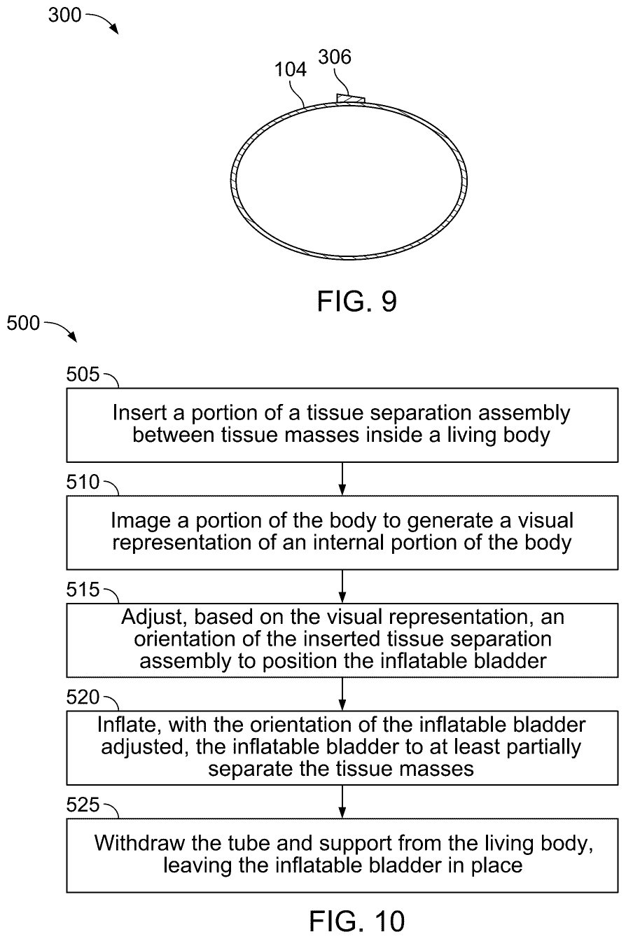

Referring also to FIG. 9, the cross-section of a support 306 of a different embodiment of a tissue separation assembly 300 can be non-symmetrical to be more easily distinguishable or visible in the axial ultrasound view. FIG. 9 illustrates a back, cross-sectional, schematic view of the tissue separation assembly 300 taken across the middle of the inflatable bladder 104. The support 306 can have a side thicker than its other side, forming a sloping cross-section. The non-symmetrical cross section of the support 306 can help a medical practitioner more easily identify the support 306 in the ultrasound imaging. For example, the non-symmetrical cross section of the support 306 can be more easily identifiable in the visual representation as compared to a support with a symmetrical cross-section when there are multiple bright, elongated spots in the ultrasound that have generally symmetrical shapes.

Referring back to FIGS. 7 and 8, the position of the inflatable bladder 104 can be adjusted in stages inside the body by partially inflating the inflatable bladder 104 and adjusting the inflatable bladder 104 until the inflatable bladder 104 is fully inflated and in its optimal position and location. For example, the inflatable bladder 104 can be first inflated to a partially inflated state (see FIG. 2). In a partially inflated state, the inflatable bladder 104 can be positioned at the desired location and orientation before continuing to inflate the inflatable bladder 104. Once the inflatable bladder 104 is at the correct location, the inflatable bladder 104 can be inflated to a fully inflated state, as shown in FIGS. 1 and 6-8. The inflatable bladder 104 can be inflated to multiple (e.g., three, four, or more) partially inflated levels and moved in between inflated levels to allow optimal positioning of the inflatable bladder 104. In some aspects, the inflatable bladder 104 can be fully inflated before any orientation or positioning of the inflatable bladder 104. For example, the inflatable bladder 104 can be fully inflated inside the living body but away from the tissue interface or away from the optimal location. With the inflatable bladder 104 fully inflated, the inflatable bladder 104 can then be moved into the tissue interface or desired location.

In some aspects, to orient the inflatable bladder 104, the inflatable bladder 104 can be deflated inside the human body during the placement procedure. For example, as shown in FIG. 8, if the practitioner placing the inflatable bladder 104 determines, based on the visual representation 401, that the inflated bladder 104 is in an incorrect position (see FIG. 8), the practitioner can partially or full deflate the inflatable bladder 104 to allow repositioning of the inflatable bladder 104. Specifically, before withdrawing the tube and support from the inflatable bladder 104, the medical practitioner can determine, based on the visual representation 401, that the inflated bladder 104 is not in the correct position. Then, the medical practitioner can deflate the inflatable bladder 104, readjust the position of the inflatable bladder, and then reinflate, with the orientation of the inflatable bladder 104 readjusted, the inflatable bladder 104.

FIG. 10 shows a flow chart of an example method 500 of separating tissue masses using a tissue separation assembly, such as the tissue separation assembly 100 of FIG. 1. The method 500 includes inserting a portion of a tissue separation assembly between tissue masses inside a living body (505). The inserting is performed by placing an inflatable bladder of the tissue separation assembly into an interface between the tissue masses with a tube fluidly coupled to the inflatable bladder. The tissue separation assembly includes a support coupled to the tube. The support extending away from the tube along one side of the inflatable bladder. The method also includes imaging a portion of the body to generate a visual representation of an internal portion of the body (510). The visual representation shows the support as visually distinguishable from the inflatable bladder. The method also includes adjusting, based on the visual representation, an orientation of the inserted tissue separation assembly to position the inflatable bladder (515). The method also includes inflating the inflatable bladder to at least partially separate the tissue masses (520). Lastly, the method includes withdrawing the tube and support from the living body, leaving the inflatable, sealed bladder in place (525).

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

A number of implementations have been described. Nevertheless, it will be understood that various modifications may be made without departing from the spirit and scope of the disclosure. For example, example operations, methods, or processes described herein may include more steps or fewer steps than those described. Further, the steps in such example operations, methods, or processes may be performed in different successions than that described or illustrated in the figures. Accordingly, other implementations are within the scope of the following claims.

What is claimed is:

1. A method of separating tissue masses, the method comprising:
inserting a portion of a tissue separation assembly between tissue masses inside a living body, by placing an inflatable bladder of the tissue separation assembly into an interface between the tissue masses with a tube fluidly coupled to the inflatable bladder, the tissue separation assembly including a support coupled to the tube and extending away from the tube along one side of the inflatable bladder;
imaging a portion of the body to generate a visual representation of an internal portion of the body, the visual representation showing the support as visually distinguishable from the inflatable bladder;
adjusting an orientation of the inserted tissue separation assembly, based on the visual representation, to position the inflatable bladder;
inflating the inflatable bladder to at least partially separate the tissue masses; and then
withdrawing the tube and support from the living body, leaving the inflatable bladder in place.

2. The method of claim 1, wherein the support comprises a visual marker and the adjusting comprises adjusting, using the visual marker as a reference point in the visual representation, the position of the inflatable bladder to center the inflatable bladder with respect to the tissue masses.

3. The method of claim 1, wherein the support is movable radially along a first axial plane of the tube and fixed laterally along a second axial plane of the tube orthogonal with respect to the first axial plane, the support releasably coupled to the inflatable bladder and configured to be displaced along the first axial plane with the inflatable bladder as the inflatable bladder is inflated, and wherein adjusting the orientation of the tissue separation assembly comprises moving the tube to move the support and the inflatable bladder with the tube as the support and the inflatable bladder are aligned with the first axial plane.

4. The method of claim 3, wherein the adjusting comprises moving the inflatable bladder together with the support while the support restricts a lateral displacement of the inflatable bladder along the second axial plane, the support allowing the inflatable bladder to inflate and expand along the first axial plane and the second axial plane.

5. The method of claim 4, wherein the support comprises a proximal end attached to the tube and a distal end releasably coupled to an end of the inflatable bladder, and wherein the adjusting comprises moving the inflatable bladder while the support maintains a radial orientation of the inflatable bladder with respect to a central axis of the tube.

6. The method of claim 1, wherein the support is made of a metal visible in ultrasound imaging and X-ray imaging, and wherein the imaging comprises ultrasound imaging or X-ray imaging.

7. The method of claim 1, wherein the imaging comprises generating an axial view of the tissue masses utilizing a transrectal ultrasound probe, the axial view showing a cross-section orthogonal with respect to a central longitudinal axis of the transrectal ultrasound probe.

8. The method of claim 1, wherein the support has a non-symmetrical cross-section, and wherein the adjusting comprises adjusting, using the non-symmetrical cross-section as a reference, the position of the support to adjust the position of the inflatable bladder.

9. The method of claim 1, wherein adjusting the orientation of the inserted tissue separation assembly comprises adjusting, after the inflatable bladder is fully inflated, the inserted tissue separation to position the inflatable bladder.

10. The method of claim 1, wherein the tissue masses comprise soft tissue masses that reside in an anatomic target location inside the body, and wherein the method further comprises, after inflating the inflatable bladder, withdrawing the tube and the support from the body, leaving the inflatable bladder in the anatomic target location inflated inside the body.

11. The method of claim 10, wherein the anatomic target location comprises a location between prostate tissues and rectum tissues.

12. The method of claim 1, wherein the imaging a portion of the body comprises generating the visual representation on an electronic screen.

13. The method of claim 1, wherein the support comprises an elongated arm having one end attached to the tube, the arm extending parallel to the tube across at least a portion of the inflatable bladder, and the adjusting comprises adjusting the orientation of the inserted tissue separation assembly by moving the tube as the arm remains parallel to the tube and attached to the tube and the inflatable bladder.

14. The method of claim 1, further comprising, before adjusting the orientation of the inserted tissue separation assembly, inflating the inflatable bladder to a partially inflated state, wherein the adjusting comprises adjusting, with the inflatable bladder in the partially inflated state, the position of the inflatable bladder, and inflating the inflatable bladder to at least partially separate the tissue masses comprises inflating the inflatable bladder to a fully inflated state.

15. The method of claim 1, further comprising:
before withdrawing the tube and support from the living body, determining, based on the visual representation, that the inflated inflatable bladder is not in the correct position;
deflating the inflatable bladder;
readjusting, based on the visual representation, the position of the inflatable bladder; and
reinflating, with the orientation of the inflatable bladder adjusted, the inflatable bladder.

16. The method of claim 1, wherein the tissue separation assembly comprises a second support, the imaging comprises generating the visual representation showing the second support as visually distinguishable from the inflatable bladder and the support, and the adjusting comprises adjusting the position of the inflatable bladder based on a location of the support and the second support in the visual representation.

17. The method of claim 1, further comprising, before withdrawing the tube and support from the living body, sealing the inflatable bladder inside the living body.

18. A tissue separation assembly, comprising:
a tube;
an inflatable bladder releasably attached and fluidly coupled to the tube; and
a support coupled to the tube and extending away from the tube along a side of the inflatable bladder;
wherein the inflatable bladder is arranged to be inserted by the tube between tissue masses inside a living body into an interface between the tissue masses, the support made of a material visually distinguishable from the inflatable bladder in a visual representation generated from body imaging of the living body, allowing an orientation of the inflatable bladder to be adjusted based on a position of the support in the visual representation.

19. A method of separating tissue masses, the method comprising:
inserting, with a tube fluidly coupled to an inflatable bladder, the inflatable bladder into an interface between tissue masses of a body;
imaging a portion of the body to generate a visual representation of an internal portion of the body, the visual representation showing a visual marker as visually distinguishable from the inflatable bladder, the visual marker coupled to and extending from the tube over a side of the inflatable bladder;
adjusting, based on the visual representation, an orientation of the tube to position the inflatable bladder;
inflating the inflatable bladder to at least partially separate the tissue masses; and then withdrawing the tube and visual marker from the living body, leaving the inflatable bladder in place.

20. The method of claim 19, wherein the visual marker comprises a support that moves, with the inflatable bladder as the inflatable bladder is inflated, parallel to a first axial plane of the tube, and is fixed against movement along a second axial plane of the tube orthogonal with respect to the first axial plane.
